(19) United States
(12) Patent Application Publication
(10) Pub. No.: US 2025/0040847 A1
(43) Pub. Date: Feb. 6, 2025

(54) PSYCHOLOGICAL EXAM SYSTEM BASED ON ARTIFICIAL INTELLIGENCE AND OPERATION METHOD THEREOF

(71) Applicant: OMNICONNECT CORP., Seoul (KR)

(72) Inventor: Yeong Jun YANG, Seoul (KR)

(21) Appl. No.: 18/580,998

(22) PCT Filed: Jul. 15, 2022
(86) PCT No.: PCT/KR2022/010345

§ 371 Date: Oct. 15, 2024

(30) Foreign Application Priority Data
Jul. 20, 2021 (KR) 10-2021-0094604

(51) Int. Cl. A61B 5/16; G06V 10/774; G06V 20/50; G06V 40/18; G16H 50/20

(52) U.S. Cl. CPC A61B 5/163; A61B 5/167; G06V 10/774; G06V 20/50; G06V 40/193; G16H 50/20; G06V 2201/03

(57) ABSTRACT

A method of operating a psychological exam system based on artificial intelligence is disclosed. The operation method of the present disclosure may include sequentially providing psychological exam content in different stimulus styles with different detection sensitivities for each of a plurality of personality factors, and acquiring eye tracking data for each of the provided psychological exam content through a camera, extracting eye movement features for the psychological exam content in different stimulus styles, respectively, based on the acquired eye tracking data, and outputting characteristic data for a plurality of personality factors according to each of the extracted eye movement features based on learning data accumulated by machine learning, and providing psychological exam result data combined with the output characteristic data for the plurality of personality factors.

### TECHNICAL FIELD

The present disclosure relates to a psychological exam system based on artificial intelligence and a method of operating the same, and more particularly, to a system that tracks a user's eye gaze and performs a psychological exam based on artificial intelligence and a method of operating the same.

### BACKGROUND ART

In psychology, self-report questionnaires and projective exams (Rorschach inkblot exam, thematic apperception exam, drawing exam, sentence completion exam, etc.) have traditionally been used to measure and evaluate personality, but self-report questionnaires are exposed to the fundamental limitation of ‘response distortion (faking)’ by the examinee, and projective exams have low reliability and validity, and have a disadvantage in that responses are strongly influenced by situational factors.

In order to overcome those fundamental limitations of existing personality exams, theories based on neuroscience and evolutionary biology have emerged, in which at least personality factors related to emotions must be interpreted by reducing them to neurotransmitters at the neural level, and emergent personality factors that cannot be explained by the emotions must be explained by using self-report questionnaires or projective exams, and in addition to the above neuroscientific developments, the fourth industrial revolution technologies represented by artificial intelligence, machine learning, and big data are being introduced in earnest into psychological evaluation and psychotherapy.

However, in order to evaluate and explain personality by reducing it to the level of molecular biology and genetics, expensive equipment and facilities such as genetic analysis equipment are required, and there are limitations that cannot be implemented on a large scale in a non-face-to-face situation.

### DISCLOSURE OF INVENTION

### Technical Problem

An aspect of the present disclosure is to provide a psychological exam system and a method of operating the same that are highly accessible and can increase validity and reliability by evaluating personality through cognitive and neural structural results such as eye movements.

### Technical Solution

A method of operating a psychological exam system based on artificial intelligence according to an embodiment of the present disclosure may include sequentially providing psychological exam content in different stimulus styles with different detection sensitivities for each of a plurality of personality factors, and acquiring eye tracking data for each of the provided psychological exam content through a camera, extracting eye movement features for the psychological exam content in different stimulus styles, respectively, based on the acquired eye tracking data, and outputting characteristic data for a plurality of personality factors according to each of the extracted eye movement features based on learning data accumulated by machine learning, and providing psychological exam result data combined with the output characteristic data for the plurality of personality factors.

Here, the plurality of personality factors may include a personality factor measured relatively sensitively according to an emotional task, a personality factor measured relatively sensitively according to a cognitive style task, and a personality factor measured relatively sensitively according to an anti-saccade task.

Furthermore, the acquiring of the eye tracking data may provide image-based psychological exam content for emotional stimulus for the personality factor measured relatively sensitively according to the emotional task, provide image and text-based psychological exam content for information processing to determine the preference of object and verbal styles for the personality factor measured relatively sensitively according to the cognitive style task, and provide target image-based psychological exam content to induce an eye movement for the personality factor measured relatively sensitively according to the anti-saccade task.

Furthermore, the personality factor measured relatively sensitively according to the emotional stimulus task may include neuroticism, extraversion, and agreeableness, and the personality factor measured relatively sensitively according to the cognitive style task may include extraversion, openness, agreeableness, and conscientiousness, and the personality factor measured relatively sensitively according to the anti-saccade task may include honesty.

Furthermore, the operation method may further include learning characteristic data for a plurality of personality factors according to eye movement features by machine learning based on characteristic data for each of the personality factors acquired by a psychological exam previously implemented through a questionnaire and training data using the extracted eye movement features as labels.

On the other hand, a psychological exam system may include at least one memory that stores a program for a psychological exam, and at least one processor that controls to sequentially provide psychological exam content in different stimulus styles with different detection sensitivities for each of a plurality of personality factors, acquire eye tracking data for each of the provided psychological exam content through a camera, extract eye movement features for the psychological exam content with different stimulus styles, respectively, based on the acquired eye tracking data, and output characteristic data for a plurality of personality factors according to each of the extracted eye movement features based on learning data accumulated by machine learning, and provide psychological exam result data combined with the output characteristic data for the plurality of personality factors.

Here, the plurality of personality factors may include a personality factor measured relatively sensitively according to an emotional task, a personality factor measured relatively sensitively according to a cognitive style task, and a personality factor measured relatively sensitively according to an anti-saccade task.

Furthermore, the at least one processor may control to provide image-based psychological exam content for emotional stimulus for the personality factor measured relatively sensitively according to the emotional task, provide image and text-based psychological exam content for information processing to determine the preference of object and verbal styles for the personality factor measured relatively sensitively according to the cognitive style task, and provide target image-based psychological exam content to induce an eye movement for the personality factor measured relatively sensitively according to the anti-saccade task.

Furthermore, the personality factor measured relatively sensitively according to the emotional stimulus task may include neuroticism, extraversion, and agreeableness, and the personality factor measured relatively sensitively according to the cognitive style task may include extraversion, openness, agreeableness, and conscientiousness, and the personality factor measured relatively sensitively according to the anti-saccade task may include honesty.

Furthermore, the at least one processor may control to learn characteristic data for a plurality of personality factors according to eye movement features by machine learning based on characteristic data for each of the personality factors acquired by a psychological exam previously implemented through a questionnaire and training data using the extracted eye movement characteristics as labels.

A computer program product according to an embodiment of the present disclosure may include a recording medium on which a program for executing the method of operating the psychological exam system is stored.

### Advantageous Effects

According to various embodiments of the present disclosure as described above, personality may be evaluated through emotional, cognitive, and neurostructural outcomes such as eye movements that are highly accessible and can be economically implemented using smartphones or PCs, thereby fundamentally eliminating the problems of response distortion in self-report exams, and low validity and reliability in projective exams.

### BRIEF DESCRIPTION OF THE DRAWINGS

FIG. 1 is a diagram for explaining a psychological exam system according to an embodiment of the present disclosure.

FIG. 2 is a flowchart showing an operation process of the psychological exam system according to an embodiment of the present disclosure.

FIG. 3 is a flowchart showing a method of operating a psychological exam system server according to an embodiment of the present disclosure.

FIG. 4 is a diagram showing psychological exam content for measuring personality factors according to an emotional task according to an embodiment of the present disclosure.

FIG. 5 is a diagram showing psychological exam content for measuring personality factors according to a cognitive style task according to an embodiment of the present disclosure.

FIG. 6 is a diagram showing psychological exam content for measuring personality factors according to an anti-saccade task according to an embodiment of the present disclosure.

FIG. 7 is a block diagram showing a configuration of a psychological exam system according to an embodiment of the present disclosure.

### BEST MODE FOR CARRYING OUT THE INVENTION

Hereinafter, preferred embodiments of the present disclosure will be described in detail with reference to the accompanying drawings. Here, it should be noted that identical reference numerals denote the same structural elements in the accompanying drawings. Furthermore, a detailed description of well-known functions and configurations that may obscure the subject matter of the present disclosure will be omitted.

Some embodiments of the present disclosure may be represented by functional block configurations and various processing steps. Some or all of those functional blocks may be implemented in various numbers of hardware and/or software configurations that perform specific functions. For example, the functional blocks of the present disclosure may be implemented by one or more microprocessors or by circuit configurations for a certain function. In addition, for example, the functional blocks of the present disclosure may be implemented in various programming or scripting languages. The functional blocks may be implemented as algorithms running on one or more processors. Additionally, the present disclosure may employ conventional technologies for electronic environment settings, signal processing, and/or data processing.

In addition, the terms such as “ . . . unit”, “module”, and the like described in this specification mean a unit that processes at least one function or operation, and may be implemented as hardware, software, or a combination of hardware and software. The “units” and “modules” are stored in an addressable storage medium and may be implemented by a program that can be executed by a processor.

For example, the “unit” and “module” may be implemented by software elements, object-oriented software elements, elements such as class elements and task elements, processes, functions, attributes, procedures, subroutines, segments of program codes, drivers, firmware, micro-codes, circuits, data, databases, data structures, tables, arrays, and variables.

Throughout the specification, in case where a portion is “connected” to the other portion, it may include a case of being “indirectly connected” to the other portion by interposing a device therebetween as well as a case of being “directly connected” to the other portion. Throughout the specification, when a portion may “include” a certain element, unless specified otherwise, it may not be construed to exclude another element but may be construed to further include other elements.

Furthermore, connection lines or connection members between elements shown in the drawings merely illustrate functional connections and/or physical or circuit connections. In an actual device, connections between elements may be represented by various replaceable or additional functional connections, physical connections, or circuit connections.

It should be noted that terms used in the present disclosure are merely used to describe specific embodiments, and are not intended to limit the present disclosure. As used herein, the singular forms are intended to include the plural forms as well, unless the context clearly indicates otherwise. Terms “include” or “have” used in the present disclosure should be understood that they are intended to indicate the presence of a feature, a number, a step, a operation, an element, a component or a combination thereof disclosed in the specification, and it may also be understood that the presence or additional possibility of one or more other features, numbers, steps, operations, elements, components or combinations thereof are not excluded in advance. That is, the description of “including” a specific configuration in the present disclosure does not exclude configurations other than the configuration, and denotes that additional configurations may be included in the implementation of the present disclosure or the scope of the technical idea of the present disclosure.

Some of the elements of the present disclosure may not be essential elements that perform essential functions in the present disclosure, but may simply be optional elements for improving performance. The present disclosure may be implemented by including only essential elements for implementing the essence of the present disclosure excluding elements used only to improve performance, and a structure including only essential elements excluding optional elements used only to improve performance is also included in the scope of rights of the present disclosure.

Hereinafter, the present disclosure will be described in more detail with reference to the accompanying drawings.

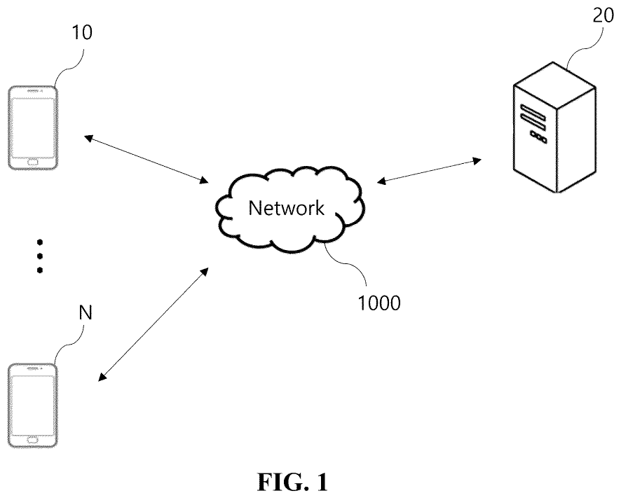

FIG. 1 is a diagram for explaining a psychological exam system according to an embodiment of the present disclosure.

Referring to FIG. 1, a psychological exam system according to an embodiment may operate by including user terminals 10 to N, a psychological exam system server 20, and a network 1000.

The user terminals 10 to N may include any device that can access the network 1000. For example, the user terminals 10 to N may include smartphones, tablets, PCs, laptops, home appliance devices, medical devices, cameras, and wearable devices. In one embodiment, the user terminals 10 to N may receive psychological exam content from the psychological exam system server 20.

In one embodiment, the user terminals 10 to N, which are terminals used by users to carry out a psychological exam on their own, may preferably be implemented as user terminals 10 to N, each mounted on a camera sensor or capable of being connected to an external camera, since the psychological exam is carried out by tracking a user's eye gaze. Accordingly, eye tracking data may be acquired by sensing the user's eye gaze through a camera sensor mounted on each of the user terminals 10 to N or an external camera connected to each of the user terminals 10 to N. Additionally, the user terminals 10 to N may transmit the eye tracking data acquired in this way to the psychological exam system server 20 through the network 1000.

The psychological exam system server 20, which is a configuration that provides psychological exam content used by the user terminals 10 to N where psychological exams are carried out, provides psychological exam content to each of the user terminals 10 to N through the network 1000. In one embodiment, the psychological exam system server 20 may include various types of servers, such as an application server, a control server, a data storage server, and a server for providing a specific function. Furthermore, the psychological exam system server 20 may process a process alone, or a plurality of servers may process the process together.

The database server (not shown) may store data necessary for the psychological exam system, wherein the database server may be part of the psychological exam system server 20, and may be operated separately from the psychological exam system server 20. In one embodiment, the psychological exam system server 20 may store information such as eye tracking data for each user and psychological exam result data.

The network 1000 may include any network that the user terminals 10 to N and the psychological exam system server 20 may access, such as the internet, an intranet, an extranet, a local area network (LAN), a metropolitan area network (MAN), and a wide area network (WAN), and the like.

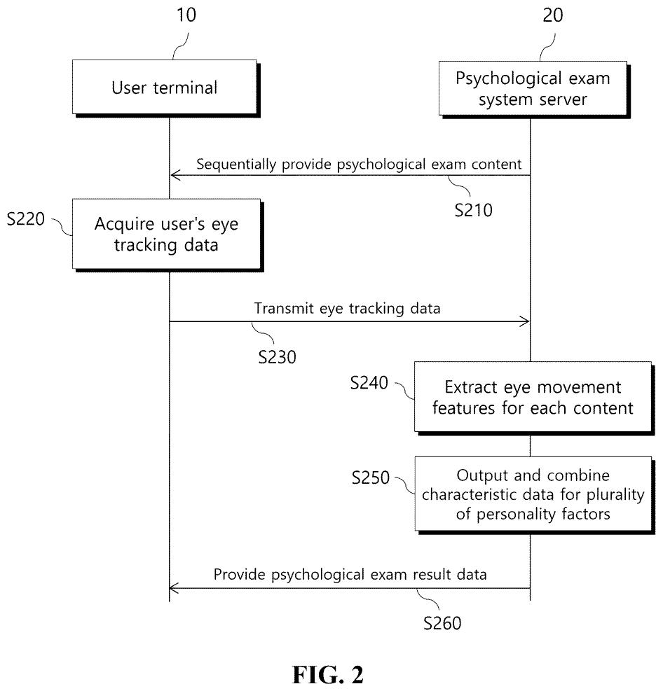

FIG. 2 is a flowchart showing an operation process of the psychological exam system according to an embodiment of the present disclosure.

Referring to FIG. 2, first, in step S210, the psychological exam system server 20 may provide psychological exam content to the user terminal 10. Here, the psychological exam content may be visual/audio content consisting of at least one of an image, a video, or a combination thereof, and may be implemented as, for example, a plurality of images or a plurality of videos.

The psychological exam system server 20 may sequentially transmit psychological exam content over time or according to an input signal received from the user terminal 10. However, according to an embodiment, the psychological exam system server 200 may transmit psychological exam content to the user terminal 10 at once, and the user terminal 10 may sequentially output the psychological exam content over time or according to an input signal received by the user terminal 10.

In the user terminal 10, psychological exam content received from the psychological exam system server 20 may be mounted on the user terminal 10 or output through an externally connected display module.

The psychological exam content may be changed after being output from the user terminal 10 for a predetermined period of time, or may be changed according to an input signal received from the user through a user interface unit mounted on the user terminal 10 or externally connected thereto. For example, when the psychological exam content includes 10 images, each image may be displayed for 5 seconds and then changed to a next image, or when the user performs a swipe or flick gesture with his or her finger on the display module, the image may be changed to the next image through touch recognition.

Meanwhile, while the psychological exam content is displayed on the user terminal 10, the user terminal 10 may sense the user's eye gaze on each psychological exam content being output through the camera sensor. Here, the camera sensor is mounted on the user terminal 10 or implemented as a camera device externally connected to the user terminal 10 to sense the user's eye gaze through photographing a direction of the user's face.

In step S220, the user terminal 10 may sense an eye movement such as a gaze direction, a movement, and a gaze duration of the user's eyeballs through the camera sensor while each psychological exam content is displayed, thereby acquiring the user's eye tracking data on each psychological exam content.

Then, in step S230, the user terminal 10 may transmit the acquired eye tracking data to the psychological exam system server 20. At this time, the user terminal 10 may transmit eye tracking data acquired while the psychological exam is carried out in real time, or may transmit all eye tracking data acquired after the psychological exam is completed.

In step S240, when the psychological exam system server 20 receives the user's eye tracking data from the user terminal 10, user eye movement features for each psychological exam content are extracted. The eye movement features extracted herein are preferably extracted based on an eye movement that is useful for detecting a plurality of personality factors that make up a psychological exam theory.

Then, in step S250, based on learning data accumulated by machine learning, characteristic data for a plurality of personality factors according to the extracted eye movement features is output, and psychological exam result data combined therewith is generated.

In step S260, the generated psychological exam result data may be provided to the user terminal 10. However, the psychological exam result data may not be provided to the user terminal 10, but may be provided by the psychological exam system server 20 itself.

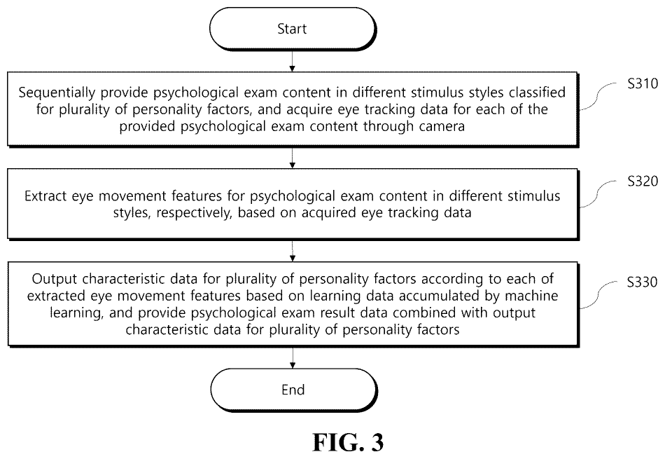

FIG. 3 is a flowchart showing a method of operating a psychological exam system server according to an embodiment of the present disclosure.

First, psychological exam content in stimulus styles with different detection sensitivities for each of the plurality of personality factors is sequentially provided, and eye tracking data for each of the provided psychological exam content is acquired through the camera (S310).

Here, the plurality of personality factors, as personality factors that make up the HEXACO model, which is a representative personality theory based on six personality factors, include extraversion, neuroticism, openness, agreeableness, conscientiousness, and honesty.

However, the plurality of personality factors herein may not be limited to personality factors according to the HEXACO model, and may be implemented in various ways, such as personality factors according to a Myers-Briggs Type Indicator (MBTI) model and the existing Big 5 model, which excludes honesty from the HEXACO model.

Meanwhile, the plurality of personality factors may be divided into personality factors measured relatively sensitively according to an emotional task, personality factors measured relatively sensitively according to a cognitive style task, and personality factors measured relatively sensitively according to an anti-saccade task, wherein the personality factors measured relatively sensitively according to the emotional task include neuroticism, extraversion, and agreeableness, the personality factors measured relatively sensitively according to the cognitive style task include extroversion, openness, agreeableness, and conscientiousness, and the personality factors measured relatively sensitively according to the anti-saccade task include honesty.

Here, the cognitive style task and the anti-saccade task may be implemented in an additional supplementary form to more accurately measure honesty, conscientiousness, and openness, which are cognitive personality factors that are relatively poorly measured according to the emotional task.

For the emotional task, image-based psychological exam content for emotional stimulus may be provided.

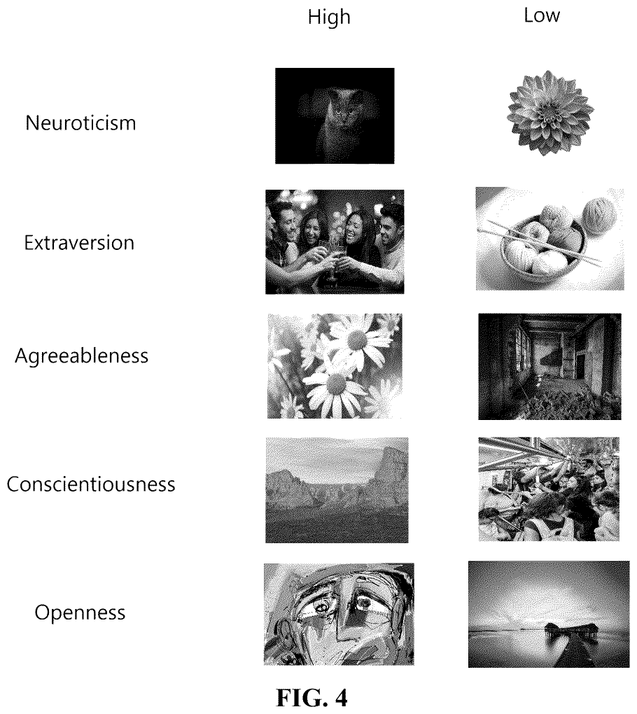

Specifically, in order to measure personality factors that make up the HEXACO model according to the emotional task, emotional images based on an emotional dimension theory may be provided as psychological exam content. In this case, the emotional images may be selected from images belonging to five types (high valence, high arousal/high valence, low arousal/low valence, high arousal/low valence, low arousal/neutral) based on the two dimensions of valence and arousal (see Table 1 and FIG. 4), or may be images or videos corresponding to anger, fear, sadness, happiness, disgust, and surprise in the basic emotion theory.

TABLE 1

| Personality factors | High | Low |
|---|---|---|
| Neuroticism | Photos with pets, lots of | Landscape, sunset, |
| | empty space in solid | waterscape, curvy visual |
| | colors (especially grey) | pattern in warm colors |
| | and people but out of | |
| | focus and in shadows | |
| Conscientiousness | People exercising, | People, pastel-colored |
| | orderly images, healthy | photos |
| | food, vegetables, natural | |
| | scenery, photos of | |
| | buildings, mountains | |
| | with sharp peaks, | |
| | zoomed-in photos, | |
| | landscape of sky, river | |
| | and sea in warm colors | |
| Openness | Moon, sky, book, | Love, cat, flowers |
| | portrait, complex shape, | |
| | landscape, abstract/ | |
| | surrealistic painting | |
| Extraversion | Amateur photography | Cat, book, knitting, |
| | of people in crowds, | flowers, plants, indoor |
| | restaurants, concerts, | photography |
| | bars, and city life | |
| Agreeableness | Flowers, warm, | Text, naked torso, black |
| | saturated colors | and white photos, hostile |
| | | pictures, grungy |
| | | background |

According to previous studies, the theme or aesthetic characteristics of a photo (a proportion of color in an image, distribution of edges, entropy, a level of details, etc.) are known to be related to personality factors, and therefore, when selecting an emotional photo, the photo may be selected using themes and aesthetic characteristics that are related to the personality factors as separate criteria than valence and arousal.

Meanwhile, while image-based psychological exam content for emotional stimulus is provided, the user terminal 10 may acquire eye tracking data through tracking the user's eye gaze, and the psychological examination system server 20 may extract eye movement features based on the acquired eye tracking data (S320).

At this time, the motion of an eye movement is expressed as three-dimensional coordinates (Xᵢ, Yᵢ, tᵢ) of a gaze point of the eye on the screen tracked by an eye tracking algorithm at a specific time tᵢ, and a number of times the gaze point is sampled per second of stimulus presentation is determined by the camera resolution. For example, a camera with a resolution of 30 Hz captures the eye 30 times per second and then determines the coordinates of the gaze point on the screen using a predetermined eye coordinate algorithm.

Then, eye movement features for detecting characteristic data for each personality factor may be calculated using the determined gaze point coordinates. Representative eye tracking features used to detect characteristic data for each personality factor may use eye movement measures such as fixation and saccade.

Fixation may be defined as an eye movement in which the gaze point on the screen is distributed within a specific spatial range (discrepancy threshold) above a minimum duration, and saccade may be defined as an eye movement that moves at a high speed (30 to 500 degrees per second) between fixations for a short period of time (30 to 80 ms).

Representative algorithms for calculating fixation and saccade from gaze point coordinates are divided into a space-based identification method (identification by discrepancy threshold, I-DT) and a velocity-based identification method (identification by velocity threshold, I-VT), and in the present disclosure, fixation and saccade are calculated by mixing the two algorithms.

Eye movement features in an emotional task may include a fixation rate (FR), a fixation duration (FD), a saccade fixation rate (SFR), a mean saccade amplitude (MSA), a mean saccade peak velocity (MSPV), a right large saccade (RLS), and a left large saccade (LLS).

Meanwhile, the method of extracting eye movement features according to an emotional task that presents an emotional stimulus as above is highly related to emotion, wherein only some personality factors (neuroticism, extroversion, agreeableness) highly related to neurotransmitter sensitivity are detected well, and personality factors highly related to a cognitive style such as honesty and conscientiousness are relatively poorly detected, thereby causing problems due to overfitting.

Therefore, in order to measure personality factors other than personality factors that are well measured according to an emotional task, psychological exam content according to the cognitive style task and anti-saccade task may be provided as an auxiliary means.

Image and text-based psychological exam content for information processing to determine the preference of object and verbal styles may be provided for the cognitive style task.

In a study that analyzed a correlation between an object-spatial imagery and verbal cognitive style and the Big 5 personality factors, it is shown that a verbal style preference has a high correlation with extroversion and openness, and an object style preference has a high correlation with conscientiousness and agreeableness.

Here, the object style may be defined as a method of processing specific and detailed imagery information on an object, the spatial style as a method of graphically expressing a relationship between concepts and mainly using a spatial relationship to explain the relationship, and the verbal style as a method of expressing a concept in language.

An object, space imagery and verbal style may be also presented as a cognitive style task to measure eye movement features therefor, thereby outputting the characteristic data of personality factors that are not well measured according to an emotional task based on a correlation between the cognitive style and the personality characteristics that have already been revealed above.

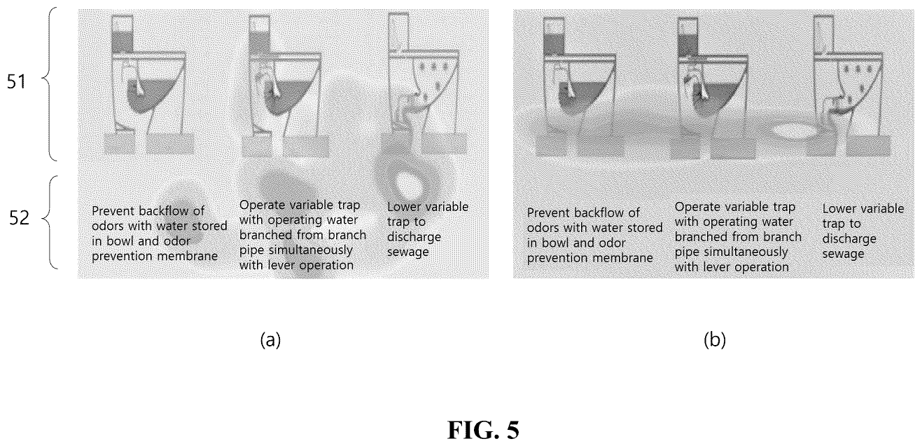

As shown in FIG. 5, the cognitive style task, which is a visual material consisting of pictures 51 and text 52 to explain the procedures, processes, or principles of a specific theme determined according to the academic area and type of knowledge, may designate an area where eyes are expected to stay in advance as an area of interest (AOI).

Two eye movement features related to AOI may be measured by using eye tracking technology. Two eye movement features associated with AOI may include a dwell time and a revisit. The dwell time may be defined as a sum of durations of all fixations and saccades passing through the AOI, and the revisit may be defined as a number of revisits since the first visit to the AOI.

For example, a user ((a) of FIG. 5) whose dwell time and revisit in a text AOI is higher than those in a picture AOI (the composite standard score is in the bottom 50%) may have personality characteristics in which extroversion and openness are high as a verbal cognitive style holder. In addition, a user ((b) in FIG. 5) whose dwell time and revisit in a picture AOI is significantly higher than those in a text area of interest (a range of the composite standard score is in the top 30%) may have personality characteristics in which conscientiousness and agreeableness are dominant as an object cognitive style holder.

Meanwhile, honesty, one of the personality factors in the HEXACO model, has the problem of being relatively difficult to measure accurately as eye movement features in emotional and cognitive style tasks. Existing research on the neurological basis of honesty has shown that the brain's dorsolateral prefrontal cortex (DLPFC) is involved in honest decision-making in decisions with high economic cost and may be the center of honesty, and the DLPFC is known to be responsible for not only a ‘value-based decision making’ function, which is related to honesty, but also a ‘context-inappropriate reaction inhibition’ function, thereby performing ‘self-control’ in an integrated manner.

In order to evaluate the ‘context-inappropriate reaction inhibition’ function of the DLPFC, an anti-saccade task is used to measure a degree of the ‘context-inappropriate reaction inhibition’ function on the subject's DLPFC in terms of the subject's response time and error rate in neuroscience.

The anti-saccade task may be explained as a task of looking at a gaze point (GP) displayed in the center of the screen, and moving one's eyes as quickly as possible in the opposite horizontal direction when another visual stimulus (VS) appears around the left and right sides of the gaze point. Here, the visual stimulus may be implemented as a target image to induce an eye movement, such as a red dot, and the like.

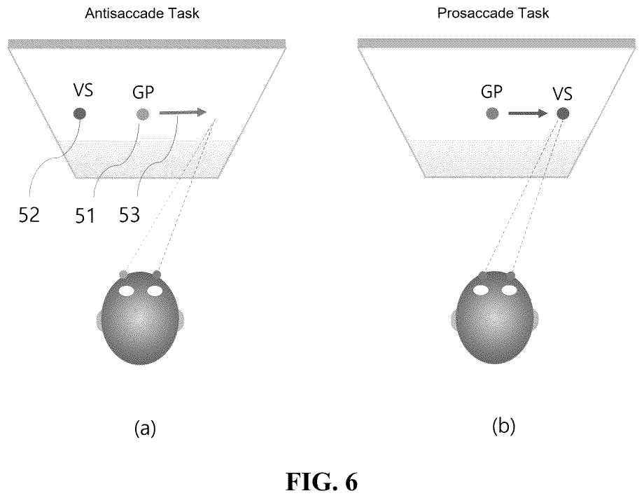

(a) of FIG. 6 shows an anti-saccade task, and (b) of FIG. 6 shows a pro-saccade task. As shown in (a) of FIG. 6, when a gaze point (GP) 51 is displayed in the center of the screen, and a visual stimulus (VS) 52 is randomly presented in the left and right visual field peripheries that are physically separated from the gaze point 51, an anti-saccade is performed in an opposite horizontal direction 53, thereby calculating a task-specific saccade reaction time (SRT) and an express saccade error (regular saccade error) as eye movement features.

Meanwhile, in the embodiment, depending on the color of the gaze point 51, the anti-saccade task and the pro-saccade task may be optionally performed. For example, it may be implemented such that when the color of the gaze point 51 is displayed as red, the user prepares to perform an anti-saccade task, and when the color of the gaze point 51 is displayed as blue, the user prepares to perform a pro-saccade task.

The faster the reaction time of the anti-saccade and the lower the error, the better the neurological function of the DLPFC to ‘inhibit a context-inappropriate behavior’, and therefore, it may be expected that the ‘value-based decision-making’ function, another aspect of self-control performed by the DLPFC, is also naturally excellent. The ‘value-based decision-making’ function is a tendency to act honestly even though the economic cost is high, that is, a tendency to avoid greed, and this tendency is classified as ‘honesty’ in the HEXACO theory.

Meanwhile, eye movement features of psychological exam content in different stimulus styles may be extracted, thereby measuring a plurality of personality factors consisting of extroversion, neuroticism, openness, agreeableness, conscientiousness, and honesty.

Specifically, characteristic data for a plurality of personality factors according to each of the extracted eye movement features may be output based on learning data accumulated by machine learning, and psychological exam result data combined with the output characteristic data for the plurality of personality factors may be provided (S330).

Meanwhile, characteristic data for a plurality of personality factors according to eye movement features may be trained by machine learning based on characteristic data for each of the personality factors acquired by a psychological exam previously implemented through a questionnaire and training data using the extracted eye movement characteristics as labels.

To this end, prior to providing psychological exam content for eye tracking, the HEXACO personality questionnaire may be carried out for a plurality of subjects to measure and group (high, middle, low) the characteristics of the plurality of subjects by personality factor. Then, a classifier may be trained in a supervised manner using training data using the grouped results through the questionnaire and the subject's eye movement features obtained later through the psychological exam content of the present disclosure as labels. The classifier used in the present disclosure may include any one of support vector machine (SVM), logistic regression (LR), and Naive Bayes (NB). In addition, a cross-validation method may be used as a supervised learning method, and subjects may be divided into a training set and a test set, and the classifier may be learned through the training set.

In a cognitive style task, when learning an algorithm to classify personality characteristic classes (high, middle, low), eye movement features according to the cognitive style task may be added to a supervised learning model as independent variables to further enhance the accuracy of the personality characteristic classification. For example, when the user who is a subject has the eye movement features of a verbal cognitive style, the user may be likely to have high extroversion and openness, and training data with the eye movement features of the verbal cognitive style as input data and openness as output data may be generated to additionally train a personality characteristic classifier.

In an anti-saccade task, when learning an algorithm to classify personality characteristics classes (high, middle, low), the eye movement features (reaction time, error rate) of the anti-saccade task may be added to the supervised learning model as independent variables to further enhance the accuracy of personality characteristic classification.

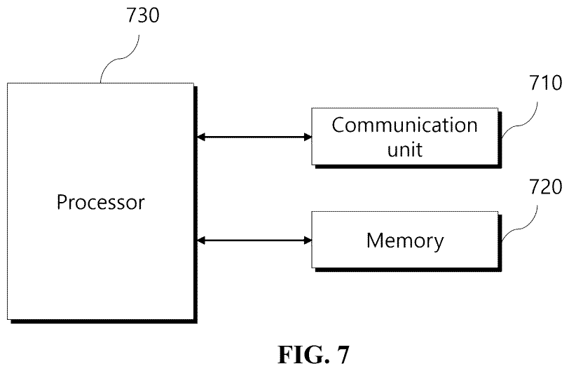

FIG. 7 is a block diagram showing a configuration of a psychological exam system according to an embodiment of the present disclosure.

As shown in FIG. 7, the psychological exam system according to one embodiment may include a communication unit 710, a memory 720, and a processor 730. However, the elements of the psychological exam system are not limited to the examples described above. For example, the psychological exam system may include more or fewer elements than those described above. In addition, the communication unit 710, the memory 720, and the processor 730 may be implemented in the form of a single chip.

The communication unit 710 may transmit and receive signals to and from an external device. The signals transmitted and received to and from the external device may include control information and data. Here, the external device may include the user terminal 10, the database server, and the like. The communication unit 710 may include both wired and wireless communication units. Besides, the communication unit 710 may receive a signal through a wired or wireless channel, output the received signal to the processor 730, and transmit the signal output from the processor 730 through the wired or wireless channel.

The memory 720 may store programs and data necessary for the operation of the psychological exam system. In one embodiment, the memory 720 may store control information or data included in signals transmitted and received by the psychological exam system. The memory 720 may be composed of a storage medium such as a ROM, a RAM, a hard disk, a CD-ROM, and a DVD, or a combination of the storage media. Additionally, there may be a plurality of memories 720. According to one embodiment, the memory 720 may store a program for performing operations for the psychological exam system according the foregoing embodiments of the present disclosure.

The processor 730 may control a series of processes in which the psychological exam system operates according to the foregoing embodiment of the present disclosure. For example, elements of the psychological exam system may be controlled to perform an operation of the psychological exam system according to one embodiment. There may be a plurality of processors 730, and the processor 730 may perform the operation of the psychological exam system through executing a program stored in the memory 720.

In one embodiment, the processor 730 may control to sequentially provide psychological exam content in different stimulus styles with different detection sensitivities for each of a plurality of personality factors, acquire eye tracking data for each of the provided psychological exam content through a camera, extract eye movement features for the psychological exam content with different stimulus styles, respectively, based on the acquired eye tracking data, and output characteristic data for a plurality of personality factors according to each of the extracted eye movement features based on learning data accumulated by machine learning, and provide psychological exam result data combined with the output characteristic data for the plurality of personality factors.

According to one embodiment, at least one processor may control to provide image-based psychological exam content for emotional stimulus for the personality factor measured according to the emotional task, provide image and text-based psychological exam content for information processing to determine the preference of object and verbal styles for the personality factor measured according to the cognitive style task, and provide target image-based psychological exam content to induce an eye movement for the personality factor measured according to the anti-saccade task.

In one embodiment, the at least one processor may control to learn characteristic data for a plurality of personality factors according to eye movement features by machine learning based on characteristic data for each of the personality factors acquired by a psychological exam previously implemented through a questionnaire and training data using the extracted eye movement characteristics as labels.

According to various embodiments of the present disclosure as described above, personality may be evaluated through emotional, cognitive, and neurostructural outcomes such as eye movements that are highly accessible and can be economically implemented using smartphones or PCs, thereby fundamentally eliminating the problems of response distortion in self-report exams, and low validity and reliability in projective exams.

Meanwhile, the foregoing embodiments may be written as a program that can be executed on a computer, and may be implemented in a general-purpose digital computer that operates the program using a computer-readable medium. In addition, a data structure used in the foregoing embodiments may be recorded on a computer-readable medium through various means. Additionally, the foregoing embodiments may be implemented in the form of a recording medium including instructions executable by a computer such as a program module executed by the computer. For example, methods implemented as software modules or algorithms may be stored in a computer-readable recording medium as codes or program commands that can be read and executed by a computer.

The computer-readable medium may be any recording medium that can be accessed by a computer, and may include volatile and non-volatile media, and removable and non-removable media. The computer-readable medium may include a magnetic storage medium such as a ROM, a floppy disk, a hard disk, and the like, and an optical readable medium such as CD-ROM, a DVD, and the like, but are not limited thereto. Furthermore, the computer-readable medium may include a computer storage medium and a communication medium.

In addition, a plurality of computer-readable recording media may be distributed over network-connected computer systems, and data stored in the distributed recording media, such as program instructions and codes, may be executed by at least one computer.

The specific implementations described in the present disclosure are merely examples, and do not limit the scope of the present disclosure in any way. For the sake of brevity of the specification, description of conventional electronic components, control systems, software, and other functional aspects of the systems may be omitted.

1-11. (canceled)

12. A method of operating a psychological exam system based on artificial intelligence, the method comprising:
sequentially providing psychological exam content for different stimulus styles with different detection sensitivities for each of a plurality of personality factors, and acquiring eye tracking data for each of the provided psychological exam content through a camera;
extracting eye movement features for the psychological exam content for the different stimulus styles, respectively, based on the acquired eye tracking data; and
outputting characteristic data for the plurality of personality factors according to each of the extracted eye movement features based on learning data accumulated by machine learning, and providing psychological exam result data combined with the outputted characteristic data for the plurality of personality factors.

13. The method of claim 12, wherein the plurality of personality factors comprise a personality factor measured relatively sensitively according to an emotional task, a personality factor measured relatively sensitively according to a cognitive style task, and a personality factor measured relatively sensitively according to an anti-saccade task.

14. The method of claim 13, wherein the sequentially providing psychological exam content comprises:
providing image-based psychological exam content for emotional stimulus for the personality factor measured relatively sensitively according to the emotional task;
providing image and text-based psychological exam content for information processing to determine a preference of object and verbal styles for the personality factor measured relatively sensitively according to the cognitive style task; and
providing target image-based psychological exam content to induce an eye movement for the personality factor measured relatively sensitively according to the anti-saccade task.

15. The method of claim 13, wherein the personality factor measured relatively sensitively according to the emotional task comprises neuroticism, extraversion, and agreeableness,
wherein the personality factor measured relatively sensitively according to the cognitive style task comprises extraversion, openness, agreeableness, and conscientiousness, and
wherein the personality factor measured relatively sensitively according to the anti-saccade task comprises honesty.

16. The method of claim 12, further comprising:
learning, by the machine learning, the characteristic data for the plurality of personality factors according to the eye movement features based on characteristic data for the each of the personality factors acquired by a psychological exam previously conducted through a questionnaire and training data using the extracted eye movement features as labels.

17. A psychological exam system, the system comprising:
at least one memory that stores a program for a psychological exam; and
at least one processor for running the program and configured to:
sequentially provide psychological exam content for different stimulus styles with different detection sensitivities for each of a plurality of personality factors, and acquire eye tracking data for each of the provided psychological exam content through a camera;
extract eye movement features for the psychological exam content for the different stimulus styles, respectively, based on the acquired eye tracking data; and
output characteristic data for the plurality of personality factors according to each of the extracted eye movement features based on learning data accumulated by machine learning, and provide psychological exam result data combined with the outputted characteristic data for the plurality of personality factors.

18. The system of claim 17, wherein the plurality of personality factors comprise a personality factor measured relatively sensitively according to an emotional task, a personality factor measured relatively sensitively according to a cognitive style task, and a personality factor measured relatively sensitively according to an anti-saccade task.

19. The system of claim 18, wherein the at least one processor is further configured to:
provide image-based psychological exam content for emotional stimulus for the personality factor measured relatively sensitively according to the emotional task;
provide image and text-based psychological exam content for information processing to determine a preference of object and verbal styles for the personality factor measured relatively sensitively according to the cognitive style task; and
provide target image-based psychological exam content to induce an eye movement for the personality factor measured relatively sensitively according to the anti-saccade task.

20. The system of claim 18, wherein the personality factor measured relatively sensitively according to the emotional task comprises neuroticism, extraversion, and agreeableness,
wherein the personality factor measured relatively sensitively according to the cognitive style task comprises extraversion, openness, agreeableness, and conscientiousness, and
wherein the personality factor measured relatively sensitively according to the anti-saccade task comprises honesty.

21. The system of claim 17, wherein the at least one processor is further configured to control learning, by the machine learning, the characteristic data for the plurality of personality factors according to the eye movement features based on characteristic data for the each of the personality factors acquired by a psychological exam previously conducted through a questionnaire and training data using the extracted eye movement characteristics as labels.

22. A computer program product comprising:
a non-transitory recording medium on which a program for executing a method of operating the psychological exam system is stored, wherein the method comprises:
sequentially providing psychological exam content for different stimulus styles with different detection sensitivities for each of a plurality of personality factors, and acquiring eye tracking data for each of the provided psychological exam content through a camera;
extracting eye movement features for the psychological exam content for the different stimulus styles, respectively, based on the acquired eye tracking data; and
outputting characteristic data for the plurality of personality factors according to each of the extracted eye movement features based on learning data accumulated by machine learning, and providing psychological exam result data combined with the outputted characteristic data for the plurality of personality factors.
